(19) United States
(12) Patent Application Publication
(10) Pub. No.: US 2014/0128766 A1
(43) Pub. Date: May 8, 2014

(54) Foley catheter

(71) Applicant: Anthony V. Beran, Yorba Linda, CA (US)

(72) Inventor: Anthony V. Beran, Yorba Linda, CA (US)

(21) Appl. No.: 13/694,193

(22) Filed: Nov. 5, 2012

(51) Int. Cl. A61M 25/00; A61B 5/01

(52) U.S. Cl. CPC A61M 25/0017; A61B 5/01
USPC 600/549; 604/544

(57) ABSTRACT

The present invention is an improved catheter, in particular, a Foley catheter. The preferred embodiment of the invention is a catheter with three lumens. The largest lumen is the drainage lumen. The side balloon lumen has a removable one-way female Luer valve threaded onto the distal end of the side balloon lumen. The removable Luer valve allows a user to drain the balloon by passive equilibrium to atmospheric pressure by removing the valve from the lumen. Alternately, the lumen can be drained by syringe. The temperature sensor lumen contains a sensor lead with a Teflon(R) outside jacket that allows the lead to more easily slide within the lumen when the lumen is stretched or retracts. The sensor lead is also preferably fixed only to the distal end of the lumen so the lead will not stretch with the lumen, but would move freely within the lumen.

### TECHNICAL FIELD

The present invention is an improved catheter, in particular, a Foley catheter.

### BACKGROUND ART

A Foley catheter is a flexible tube that is usually passed through the urethra and into the bladder. The tube has two or more separated channels, or lumens, running down its length. One lumen is open at both ends, and allows urine to drain out into a collection bag. Another lumen, or side lumen, typically has a valve on the outside end and connects to a balloon at the tip; the balloon is inflated with sterile water when it lies inside the bladder, in order to stop it from slipping out. Foley catheters are commonly made from silicone rubber or natural rubber. A temperature, or thermistor, sensor can be placed inside one of the Foley catheter's side lumen with a sensing tip positioned at the catheter's proximal end. The proximal end is usually beveled for atraumatic insertion. The temperature sensor is used to measure a patient's bladder or core temperature.

The prior art Foley catheters with temperature sensors can develop irregularities and bumps on the surface of the catheter as the catheter is removed from a patient. The force on the catheter being pulled from a patient tends to stretch the elastic catheter material. When the catheter is released, the wire of the temperature sensor do not release evenly with the elastic catheter material. Instead, the wire, either attached along the length of the lumen or frictionally engaged along the length of the lumen, bunches at points along the length of the lumen. The irregularities and bumps caused by this uneven response to a pull on the catheter can make removal of the catheter more difficult and can cause urethral abrasion.

Another issue with prior art Foley catheters is the removal of the sterile water from the catheter retention balloon without creating creases on the surface of the balloon and without leaving water inside the balloon. Either condition can result in difficult catheter withdrawal and possible urethral injury. Typically, there is a one-way Luer valve used for balloon inflation permanently attached to the catheter's inflation lumen. The fluid from the balloon is removed by applying a negative pressure to the Luer valve, usually with a syringe. However, the uncontrolled application of negative pressure can result in fluid retention within the balloon if insufficient pressure is applied or crease on the balloon's surface when excessive pressure is applied.

### SUMMARY OF THE INVENTION

The present invention is an improved catheter, in particular, a Foley catheter. The preferred embodiment of the invention is a catheter with three lumens. The largest lumen is the drainage lumen. The side balloon lumen has a removable one-way female Luer valve threaded onto the distal end of the side balloon lumen. The removable Luer valve allows a user to drain the balloon by passive equilibrium to atmospheric pressure by removing the valve from the lumen. Alternately, the lumen can be drained by syringe. The temperature sensor lumen contains a sensor lead with a Teflon(R) outside jacket that allows the lead to more easily slide within the lumen when the lumen is stretched or retracts. The sensor lead is also preferably fixed only to the distal end of the lumen so the lead will not stretch with the lumen, but would move freely within the lumen.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings.

FIG. 1 is a top perspective view of a preferred embodiment of the invention;

FIG. 2 is a cross-sectional view of the catheter and the lumens;

FIG. 3 is a side perspective view of the temperature sensor lead;

FIG. 4 is a side cross-sectional view of the proximate end of the temperature sensor lead;

FIG. 5 is a cross-sectional view of the temperature sensor lead;

FIG. 6 is a side perspective view of the temperature sensor connector;

FIG. 7 is a top perspective view of a preferred embodiment of the catheter with the temperature sensor lead attached to the lumen at the distal end of the side sensor lumen;

FIG. 8 is a top perspective view of an alternative embodiment of the catheter with the temperature sensor lead attached to the lumen at the distal and proximate end of the side sensor lumen;

FIG. 9 is a top perspective view of a preferred embodiment of the invention with the luer valve disconnected from the side balloon lumen; and,

FIG. 10 is a top cross-sectional view of a preferred embodiment of the proximal end of the catheter.

### DETAILED DESCRIPTION OF THE PREFERRED EMBODIMENTS

Various embodiments are now described with reference to the drawings, wherein like reference numerals are used to refer to like elements throughout. In the following description, for purposes of explanation, numerous specific details are set forth in order to provide a thorough understanding of one or more embodiments. It may be evident, however, that such embodiment(s) may be practiced without these specific details.

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s). The following description is provided to enable any person skilled in the art to make and use the invention and sets forth the best modes contemplated by the inventors of carrying out their invention. Various modifications, however, will remain readily apparent to those skilled in the art, since the general principles of the present invention have been defined herein specifically to provide an improved Foley catheter with temperature sensor.

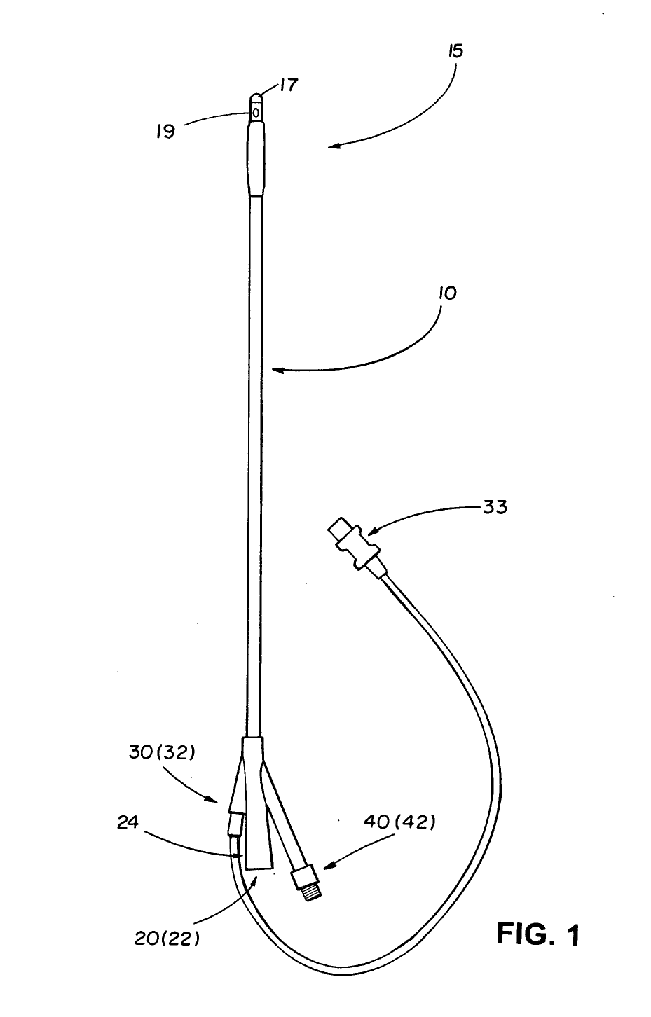

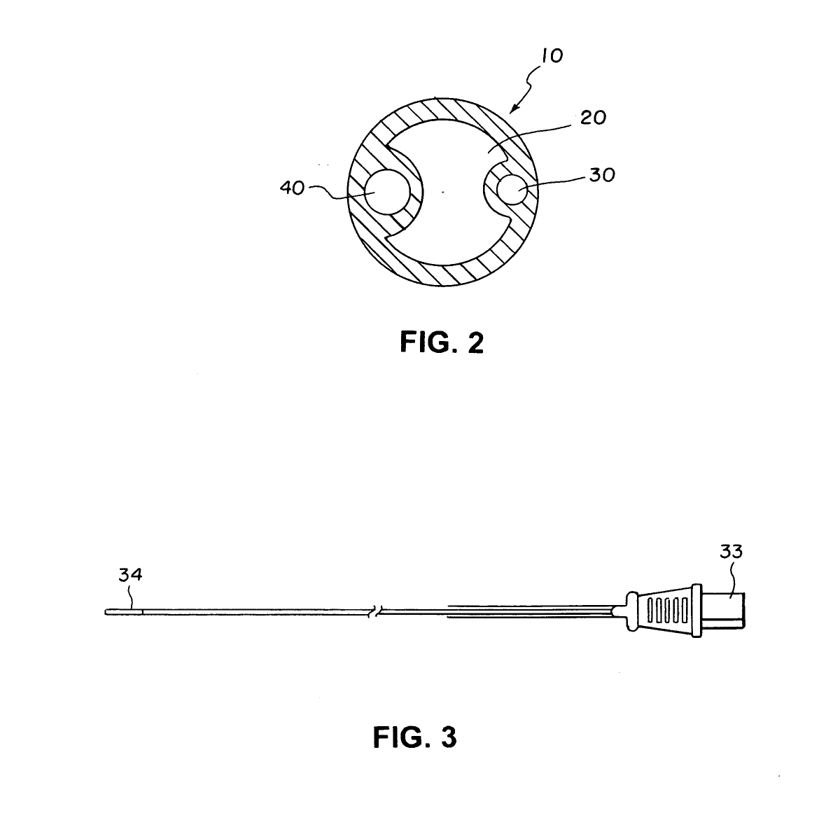

Referring now to FIG. 1, a top view of a preferred embodiment of the invention 10 is shown. Preferably, the Foley catheter 10 comprises a proximal end 15 with side drainage openings 19. The distal end of the catheter 10 preferably has three lumen endings 22, 32 and 42. Referring now to FIG. 2, a cross-section of the catheter 10 is shown. As shown in FIG. 2, the catheter 10 has three lumens 20, 30, and 40. The largest diameter lumen is preferably the drainage lumen 20 extending from the side drainage openings 19 to the drainage lumen ending 22. This lumen 20 is preferably used to drain urine from the bladder. The drainage lumen ending 22 preferably has a fitting 24 to connect the lumen 20 to a catheter bag (not shown). The catheter lumens are preferably made of silicon.

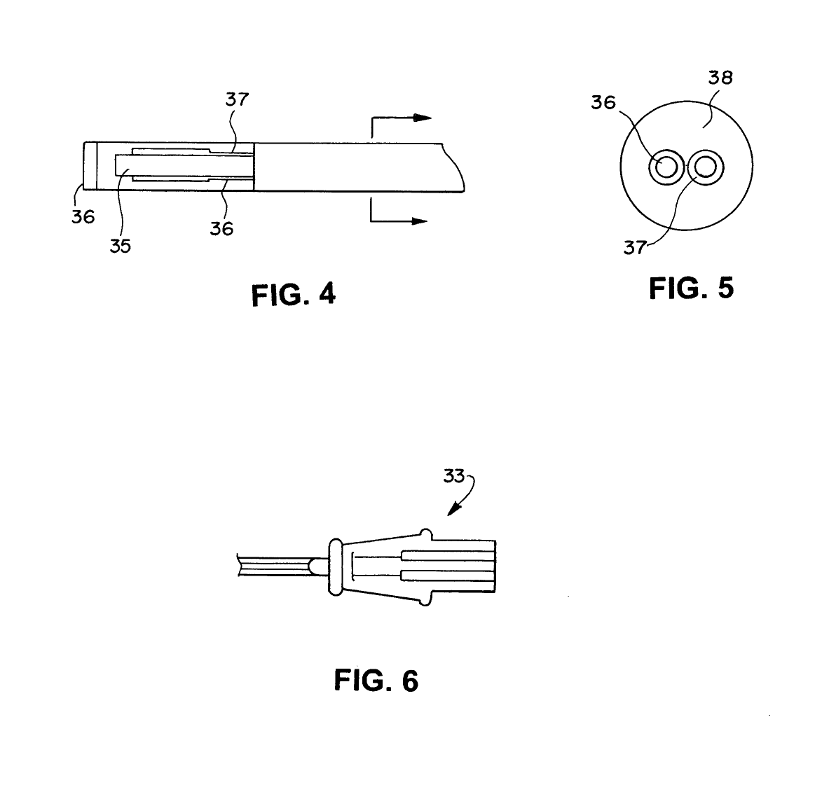

The side lumens 30 and 40 shown in FIG. 2 are preferably the side sensor lumen 30 and the side balloon lumen 40. The side sensor lumen 30 preferably extends from the sensor tip 5 to the sensor lumen ending 32. A preferred embodiment of a temperature sensor lead 34 is shown in FIG. 3. The temperature sensor lead 34 shown in FIG. 3 comprises a thermistor 35 at a tip of the lead 34. The thermistor 35 is preferably a YSI 400 series equivalent thermistor chip. As shown in FIG. 4, the thermistor 35 is placed inside of polyimide tubing 39, and it is encapsulated with an epoxy to protect the chip from outside contamination. However, other tubings or molded caps can be used to encapsulate chip 35. Referring to FIG. 5, the thermistor 35 is connected to two wires 36, 37 that extend inside the length of the lead 34. The wires 36, 37 preferably are made of 32AWG copper wire and have Teflon® wire insulation and are inserted into an outside jacket 38, preferably made of Teflon® as well or any other material with low adhesion properties, e.g. nylon. The lead 34 shown in FIG. 3 ends in a molex connector 33 as shown in FIG. 6. However, other connectors such as a 3 mm pin jack can be used.

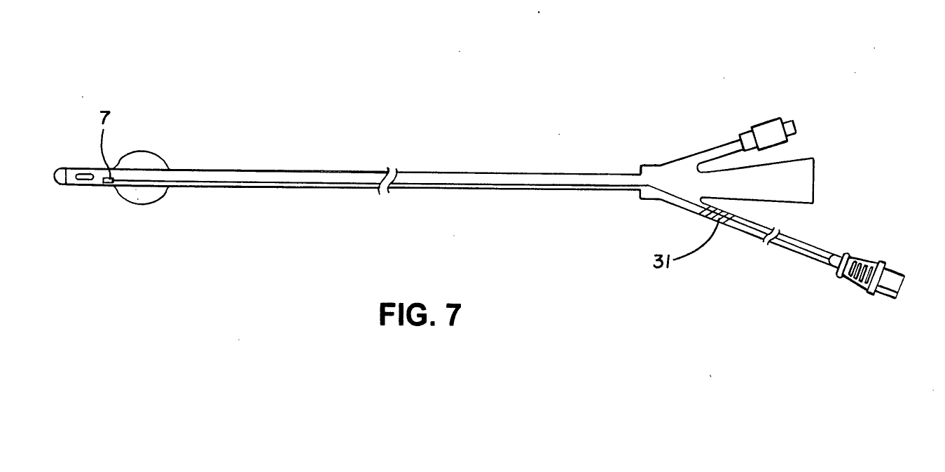

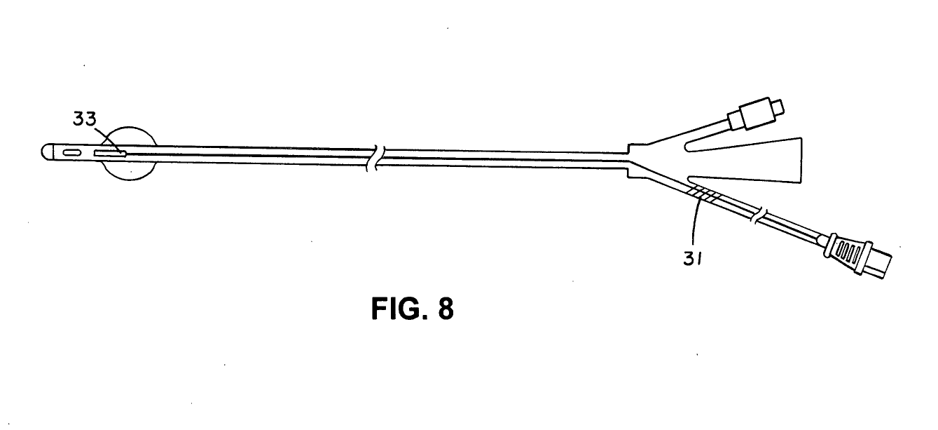

In the preferred embodiment shown in FIG. 7, the temperature sensor lead 34 is attached to the catheter 10 only at the distal end 31. The lead 34 is attached at distal end 31 by using epoxy suitable for bonding to silicon. The length of the lead 34 from the distal end 31 to the proximal end is free to slide within the side sensor lumen 30. By using Teflon® or other friction-reducing materials, the lead 34 does not resist the stretching and contracting of the catheter 10 during insertion or removal. Accordingly, the risk of bumps or irregularities on the surface of the catheter 10 is reduced. In an alternate embodiment shown in FIG. 8, the temperature sensor lead 34 is fixed to the side sensor lumen 30 at both the proximate 33 and distal 31 ends. In the FIG. 8 embodiment, the stiffness of the lead 34 resists stretching and contracting in the catheter 10 to reduce the risk of bumps or irregularities on the surface of the catheter 10.

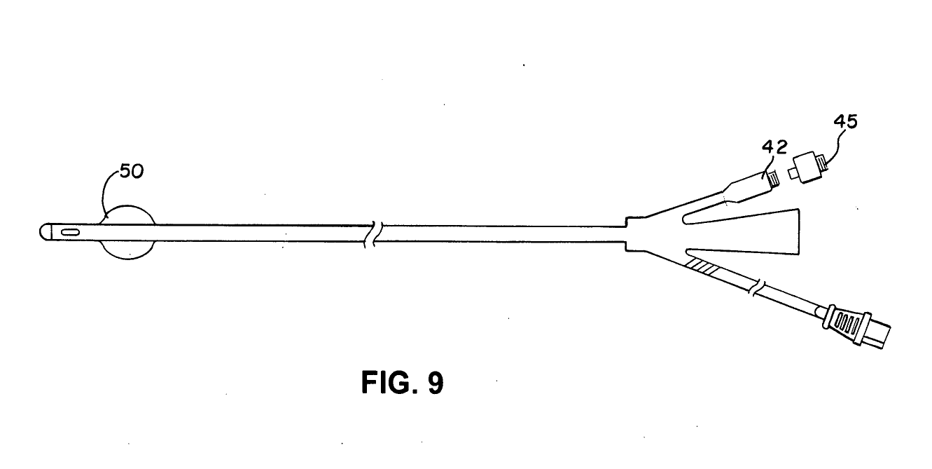

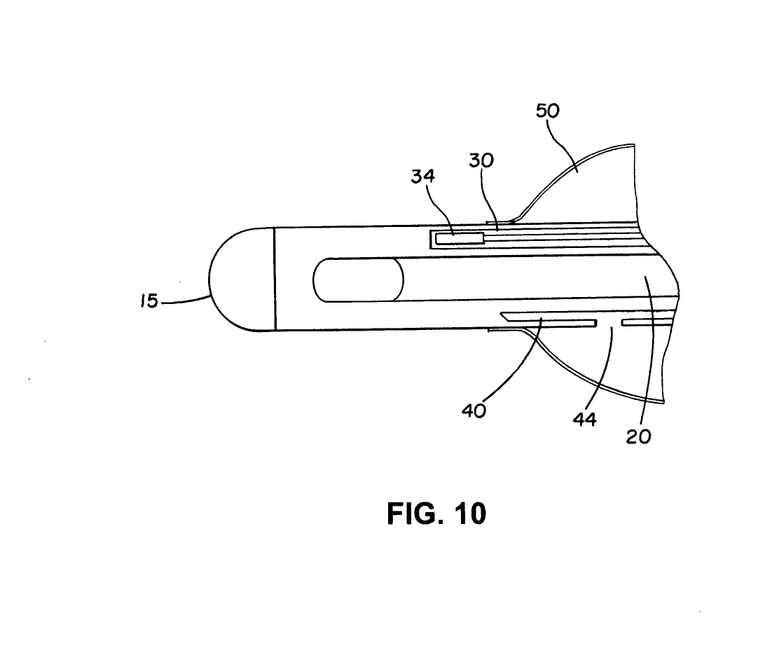

Referring now to FIG. 9, the side balloon lumen 40 extends from a balloon 50 to the balloon lumen ending 42. The balloon 50 in FIG. 9 is shown inflated. The balloon 50 is preferably inflated by sterile water. The side balloon lumen 40 preferably has a balloon inflation opening 44 in communication with the balloon 50 as shown in FIG. 10. The sterile water travels down the side balloon lumen 40 through the opening 44 and into the balloon 50 to inflate the balloon 50. The water is held within the side balloon lumen 40 by a one-way removable Luer valve 45 attached to the balloon lumen ending 42, which in the embodiment shown is a female Luer lock connector. Preferably, the valve 45 is threaded onto the ending 42 and is a male to female connector/adapter that can receive a syringe (not shown) for inflation and deflation of the balloon 50. Also, the valve 45 can be removed to allow the balloon 50 to drain via passive equilibrium to atmospheric pressure as opposed via negative pressure applied by a syringe. Also, the fluid can be removed from the balloon by applying negative pressure using a syringe, followed by the removal of the valve 45 to complete the equilibration to atmosphere. This assures a user that the balloon 50 is fully drained of water without creating creases in the balloon 50 due to excessive negative pressure or incomplete deflation, because not enough pressure was applied. The interconnection between valve 45 and fitting 42 can incorporate a safety lock against accidental disconnect.

Thus, an improved Foley catheter with a temperature sensor is described above that resists surface deformation during insertion and extraction and can be more certainly drained without over-deflating the balloon. In each of the above embodiments, the different positions and structures of the present invention are described separately in each of the embodiments. However, it is the full intention of the inventor of the present invention that the separate aspects of each embodiment described herein may be combined with the other embodiments described herein. For example, the features described above can be used in catheters with two, three or more lumens. Those skilled in the art will appreciate that adaptations and modifications of the just-described preferred embodiment can be configured without departing from the scope and spirit of the invention. Therefore, it is to be understood that, within the scope of the appended claims, the invention may be practiced other than as specifically described herein.

Various modifications and alterations of the invention will become apparent to those skilled in the art without departing from the spirit and scope of the invention, which is defined by the accompanying claims. It should be noted that steps recited in any method claims below do not necessarily need to be performed in the order that they are recited. Those of ordinary skill in the art will recognize variations in performing the steps from the order in which they are recited. In addition, the lack of mention or discussion of a feature, step, or component provides the basis for claims where the absent feature or component is excluded by way of a proviso or similar claim language.

While various embodiments of the present invention have been described above, it should be understood that they have been presented by way of example only, and not of limitation. Likewise, the various diagrams may depict an example architectural or other configuration for the invention, which is done to aid in understanding the features and functionality that may be included in the invention. The invention is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the present invention. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the invention, whether or not such embodiments are described and whether or not such features are presented as being a part of a described embodiment. Thus the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term ^{“}example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, may be combined in a single package or separately maintained and may further be distributed across multiple locations.

As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A Foley catheter comprising:
a drainage lumen; and,
a side lumen having a proximal end and a distal end, the side lumen containing a temperature sensor lead where the lead is only attached to the side lumen at the distal end such that the lead can slide within the side lumen when the side lumen stretches or contracts.

2. The Foley catheter of claim 1 where the temperature sensor lead has a wire with a layer of TEFLON® insulation.

3. The Foley catheter of claim 2 where the wire further comprises a TEFLON® outside jacket encasing the layer such that the lead has reduced frictional contact with the side lumen.

4. The Foley catheter of claim 1 where the temperature sensor lead further comprises a connector extending from the distal end of the side lumen.

5. A Foley catheter comprising:
a drainage lumen; and,
a side lumen having a proximal end and a distal end, the side lumen containing a temperature sensor lead with a temperature sensor and having stiffness where the lead is attached to the side lumen at the proximal end at or below the temperature sensor and at the distal end, such that the stiffness of the lead resists stretching of the side lumen and the catheter.

6. A Foley catheter comprising:
a drainage lumen; and,
a side lumen having a proximal end and a distal end, where the side lumen is connected to a balloon at the proximal end and a female Luer lock connector at the distal end connected to a removable one way Luer valve connector/adapter.

7. The Foley catheter of claim 6 where the removable one-way Luer valve is threaded and the distal end of the side lumen is threaded.

8. A Foley catheter comprising:
a drainage lumen;
a side sensor lumen having a proximal end and a distal end, the sensor lumen containing a temperature sensor lead where the lead is only attached to the side lumen at the distal end such that the lead can slide within the side lumen when the side lumen stretches or contracts; and,
a side balloon lumen having a proximal end and a distal end, where the balloon lumen is connected to a balloon at the proximal end and a removable one-way female Luer valve threaded to the distal end of the side balloon lumen.

9. The Foley catheter of claim 8 where the temperature sensor lead has a wire with a layer of TEFLON® insulation.

10. The Foley catheter of claim 8 where the wire further comprises a TEFLON® outside jacket encasing the layer such that the lead has reduced frictional contact with the side lumen.
